(19) United States
(12) Patent Application Publication
(10) Pub. No.: US 2014/0056818 A1
(43) Pub. Date: Feb. 27, 2014

(54) PROCESS FOR DELIVERING ENCAPSULATED NEUTRAL BIOIMAGING MOLECULES, COMPLEX, AND PROCESS THEREOF

(71) Applicants: Yamuna Krishnan, Bangalore (IN); Dhiraj Bhatia, Bangalore (IN)

(72) Inventors: Yamuna Krishnan, Bangalore (IN); Dhiraj Bhatia, Bangalore (IN)

(73) Assignee: NATIONAL CENTRE FOR BIOLOGICAL SCIENCES, Bangalore, Karnataka (IN)

(21) Appl. No.: 14/114,568

(22) PCT Filed: May 9, 2012
(86) PCT No.: PCT/IB2012/052294

§ 371 Date: Oct. 29, 2013

(30) Foreign Application Priority Data
May 10, 2011 (IN) 1471/CHE/2011

(51) Int. Cl. G01N 33/50

(52) U.S. Cl. CPC G01N 33/5091
USPC 424/9.6; 435/29

(57) ABSTRACT

The present disclosure relates to delivering neutral bioimaging molecules encapsulated within icosahedral DNA capsules in vivo and in vitro. The present disclosure also discloses the entrapment of neutral bioimaging molecules like FITC dextran within the cavity of a DNA polyhedron without any molecular recognition or chemical conjugation between host (DNA icosahedron) and cargo (like FITC Dextran). This DNA polyhedron is structurally well defined and shows high encapsulation efficiency. The present disclosure also relates to complex formed due to the encapsulation of neutral bioimaging agents within icosahedral DNA capsules.

### TECHNICAL FIELD

The present disclosure relates to delivering neutral bioimaging molecules encapsulated within icosahedral DNA capsules in vivo and in vitro. The present disclosure also discloses the entrapment of neutral bioimaging molecules like FITC dextran within the cavity of a DNA polyhedron without any molecular recognition or chemical conjugation between host (DNA icosahedron) and cargo (like FITC Dextran). This DNA polyhedron is structurally well defined and shows high encapsulation efficiency. The present disclosure also relates to complex formed due to the encapsulation of neutral bioimaging agents within icosahedral DNA capsules.

### BACKGROUND AND PRIOR ART

Synthetic host scaffolds present exciting possibilities for emergent behavior given their amenability to encapsulate functional guests. In host-guest complexes, two independent entities are supramolecularly associated resulting typically in the entrapment of one of these (guest or cargo) inside the available volume of the other (host). Host guest complexes based on synthetic molecular hosts, such as cyclodextrins, calixarenes, cucurbiturils, porphyrins, crown ethers, zeolites and cryptands, form structurally well-defined, synthetic scaffolds. A general strategy to achieve encapsulation within synthetic hosts involves the controlled polymerization of host units resulting in a well defined cavity within which the guest (cargo) is accommodated and stabilized by molecular recognition. Recently, bioinspired peptide scaffolds, based on viral proteins that mimic naturally occurring hosts like viruses, have emerged as promising synthetic hosts. Controlled oligomerization of DNA motifs into well-defined polyhedra enclosing well-defined cavities has also been recently demonstrated. However the potential of DNA polyhedra as synthetic molecular hosts for encapsulating functional cargo has remained unexplored, despite the internal cavity being utilizable. Previous studies had established that DNA motifs comprising five-way junctions could undergo oligomerization resulting in a well-defined DNA icosahedron.

The current state of the art in the encapsulation involves the use of various synthetic molecules and biomolecules as encapsulating agents. These can be broadly classified as (a) structurally well defined and (b) structurally less defined.

These synthetic molecules includes synthetic hosts like cyclodextrins, crown ethers, cryptands, zeolites, etc and systems like liposomes, PLGA microspheres, protein capsules, etc which have a well defined cavity in which the guest molecules can be encapsulated by molecular recognition between the guest molecules and hosts. The limitation here is that this is amenable only to (i) those molecules bearing a recognition moiety, or (ii) where functionality of the molecule is retained after subjecting it to a chemical reaction that appends the recognition moiety.

Turberfield group (US2009227774A1) showed that by covalent attachment of Cytochrome-c to a DNA strand (one of the components of a DNA tetrahedron), the Cytochrome-c can be positioned within the tetrahedron's internal cavity. By changing the position of attachment along this DNA strand, the Cytochrome-c could be positioned on the outer surface of the DNA tetrahedron. This study uses a protein-DNA covalent conjugate that has the morphology of a host-cargo complex. It does not trap a freely floating Cytochrome-c within the tetrahedral cavity. Also, one more drawback this system suffers is that tetrahedron is a very simple system. So, if the dimensions of tetrahedron are increased to encapsulate more number of molecules, it would also simultaneously increase the associated pore size through which the encapsulated molecules would leak out.

Currently the main challenge with neutral bioimaging molecules is delivering them to different cells. Various carriers are known in the art (like liposomes, PEG based nanoparticles) to deliver neutral bioimaging molecules to in-vivo. However, the main disadvantage faced is non-uniformity and non-homogeneity of these vehicles. Further, random distribution of the delivering moieties, which often leads to their diffusion in vivo leading to mis-delivering, dissociation and degradation of the vehicles, makes the delivering of neutral bioimaging molecules difficult.

Use of particles such as liposomes and PEG nanoparticles, which are formed from a mixture of monomer units, to encapsulate labelled neutral units will lead to random diffusion of the labeled units thus making it difficult to control the delivery of the label.

Another challenge in delivering neutral bio-imaging molecules to cells is the stability of the carrier molecules and the altered uptake of the encapsulated cargo. If the cell receptors recognize carriers like liposomes, PEG based nanoparticles, etc and internalize them, an enhanced specific uptake of the neutral bio-imaging molecules could be observed. However, the stability of such carrier molecules in vivo is low, as most host liposomes are made of lipid membranes and would either integrate with cell membranes of cells or dissociate within cellular environment. This limitation of the prior art needs to be overcome, and the same is accomplished by the instant invention.

### STATEMENT OF THE DISCLOSURE

Accordingly the present disclosure relates to a method of delivering neutral bio-imaging molecule(s) to a cell, said method comprising act of encapsulating the neutral bioimaging molecule(s) within a DNA icosahedron and delivering the DNA icosahedron to the cell; a complex comprising DNA icosahedron encapsulating neutral bioimaging molecule(s); and a process for synthesising a complex comprising DNA icosahedron encapsulating neutral bioimaging molecule(s), said process comprising acts of—a) assembling DNA molecules to obtain a semi-icosahedral DNA capsule, and b) incubating the neutral bioimaging molecule(s) with the semi-icosahedral DNA capsule, and ligating the semi-icosahedral DNA capsules to obtain neutral bioimaging molecule(s)-DNA icosahedron complex, wherein the DNA icosahedron encapsulates the neutral bio-imaging molecule(s).

### BRIEF DESCRIPTION OF ACCOMPANYING FIGURES

In order that the disclosure may be readily understood and put into practical effect, reference will now be made to exemplary embodiments as illustrated with reference to the accompanying figures. The figure together with a detailed description below, are incorporated in and form part of the specification, and serve to further illustrate the embodiments and explain various principles and advantages, in accordance with the present disclosure, wherein:

FIG. 1 shows formation and characterization of host-cargo complex of DNA icosahedron and FD10 (a) Schematic illustrating the formation of FD10 loaded icosahedra (I_{FD10}). Two complementary half icosahedra VU₅ and VL₅ are mixed in a 1:1 ratio in 2 mM FD10 solution and purified from free FD10. (b) (Left) Gel electrophoretic mobility shift assay for the formation of I_{FD10}. 0.8% agarose gel (1×TAE) showing association of FD10 with icosahedron: lane 1. FD10, lane 2. 1:1 (VU₅:VL₅)+2 mM FD10 post ligation, lane 3. Purified I_{FD10}. Gel is visualized using FITC excitation. (Right) Size exclusion chromatogram (SEC-HPLC) of I_{FD10} complex post gel excision. SEC traces are followed at 254 nm (black) and 488 nm (grey). Inset: SEC of standard, reference sample of unlabeled, unloaded icosahedron I (retention time 8 min) (c) Dynamic Light Scattering (DLS) traces of free FD10 (grey squares), the standard sample of DNA icosahedra, I (open black circles) and purified I_{FD10} complex (black squares). (d) Possible modes of association of cargo (FD10) with the host DNA capsule. Also given are cartoons representing all the species used in the present study.

FIG. 2 shows the molecular characterization of the formation of I^{TMR} Gel electrophoretic mobility shift assay for the formation of I/2^{TMR} (U₅V^{TMR}) and I^{TMR}. (a) 15% native PAGE (1×TBE) showing mobility shift between V1^{TMR} and I/2^{TMR} demonstrating formation of I/2^{TMR} from V1^{TMR}: lane 1. V1^{TMR}, lane 2. I/2^{TMR} ligated, visualized by exciting TMR label. Lanes 1*, 2* show corresponding ethidium bromide staining of lanes 1 and 2. (b) 0.8% agarose gel (1×TAE) showing formation of I^{TMR} from I/2^{TMR}: lane 1. I/2^{TMR} lane 2. I/2, lane 3. I^{TMR} lane 4. I, visualized by exciting TMR label. Lanes 1*, 2*, 3* and 4* show corresponding ethidium bromide staining of lanes 1-4.

FIG. 3 shows accessibility of FD10 in the host-cargo complex and interaction of encapsulated cargo within the host capsule (a) Fluorescence intensity-based quenching assay for free FD10 (gray squares) and I_{FD10} complex (black squares). All quenchers are gold nanoparticles (GNPs) of indicated sizes except iodide (0.5 nm), TEMPO-A (1 nm) and TEMPO-Dextran (2.5 nm). Mean values of two experiments are presented, with their corresponding s.d. (b) Fluorescence lifetime measurements of free FD10 (grey squares) and I_{FD10} complex (black squares) with the same quenchers. Mean values of two independent experiments are presented, along with their s.d. Inset: The lifetimes of free (grey bar) and encapsulated (black bar) FD10 in absence of any quencher. Error bars indicate s.d. (c) Fluorescence anisotropy of free FITC, FD10, I_{FD10}, externally added FD10 to preformed I and dsDNA, and I^{FITC}. Each bar represents mean of three independent experiments, with their associated s.d. (d) Perrin plot showing rotational correlation times of FITC (black circles), FD10 (grey squares), I_{FD10} (black squares), and I^{FITC} (black triangles) (e) shows the two possibilities for entrapment of cargo wherein the cargo can be either encapsulated inside the host capsule or it is attached on the outer surface of the host capsule.

FIG. 4 shows effect of DNA cage on quenching of encapsulated cargo. Average fluorophore lifetimes of tetramethylrhodamine (TMR) in free TD10 (grey bar) and I_{TD10} (black bar) are within error, thus confirming that the DNA cage has negligible effect on the quenching of the encapsulated cargo. Error bars indicate s.d.

FIG. 5 shows the quenchers used in quench fluorescence by dynamic quenching. Plot showing A/A₀ ratio of FITC, where A and A₀ are absorbance values in presence and absence of quencher, respectively. For quenchers of all used sizes, the A/A₀ ratio of FITC is ≧1, confirming that in all cases here, quenching is purely collisional.

FIG. 6 shows FD10 in the I_{FD10} complex is not associated with the DNA scaffold, I. Fluorescence anisotropies of two different fluorophores when encapsulated inside or covalently attached to the DNA icosahedron and its component modules show similar trends, indicating that anisotropy observations are fluorophore independent. Error bars indicate s.d.

FIG. 7 shows encapsulated FD10 is uptaken by anionic ligand binding receptors in cellulo (a) Schematic showing the different pathways of endocytosis adopted by free and encapsulated FD10. (SV: spherical vesicle, EE: early endosome, LE: late endosome, Ly: lysosome) (b) I_{FD10} is endocytosed via the anionic ligand binding receptor (ALBR) pathway in hemocytes. Cells are pulsed with FD10 and I_{FD10} in the presence and absence of maleylated BSA (mBSA, 30 μM). Internalization is quantified in terms of mean fluorescence intensity of cells, normalized to the mean intensity of I_{FD10}. The contribution from autofluorescence is also shown. Error bars indicate s.e.m. (n=25 cells) (c) Epifluorescence images showing internalization of I_{FD10} in Drosophila hemocytes. Cells, that are pulsed with I_{FD10} for 5 min, chased for 5 min and imaged, show distinct punctate structures containing I_{FD10}. (Scale bar: 10 μm) (d) FD10 is internalized in hemocytes while it is present inside the DNA cage. Cells, pulsed with I_{FD10}^{TMR} chased for 5 min and imaged in the FITC channel (488 nm excitation, green) and TMR channel (554 nm excitation, red), show colocalization. (Scale bar: 10 μm).

FIG. 8 shows the uptake of I_{FD10} by coelomocytes in C. elegans. (a) Schematic of C. elegans showing the positions of coelomocytes. (Adapted from Worm Atlas) (b) Schematic showing uptake of encapsulated FD10 by coelomocytes post-injection. (c) Retention of functionality of encapsulated FD10 enabling the measurement of endosomal pH.

FIG. 9 shows I_{TMR} is uptaken by coelomocytes in C. elegans. (a) Representative image showing a wild type hermaphrodite microinjected (red arrowhead indicates point of injection) with a solution of I^{TMR} into the pseudocoelom, from where it is uptaken by coelomocytes (indicated by white arrowheads, scale bar: 100 μm). Inset shows a representative confocal image of a pair of coelomocytes typically labeled with I^{TMR} (Scale bar: 10 μm) (b) Representative colocalization of GFP and I^{TMR} in an arIs37 [pmyo-3::ssGFP] worm (transgenics where the muscles secrete soluble GFP into the pseudocoelom which is endocytosed by the coelomocytes, and is thus a reliable marker for coelomocytes) injected with 3 μM solution of I^{TMR} showing that I^{TMR} marks coelomocytes. (Scale bar: 10 μm, n=10).

FIG. 10 shows encapsulated FD10 uptaken by coelomocytes of C. elegans is functional (a) Representative image showing a wild type hermaphrodite microinjected with a solution of FD10 (upper panel) and I_{FD10} (lower panel) into the pseudocoelom, from where it is uptaken by coelomocytes. (Scale bar: 10 μm) (b) In vitro pH calibration curve of I_{FD10} obtained by dual excitation of fluorescein at 480 nm and 430 nm. Error bars indicate s.e.m of three measurements. (n=10) Inset: Fold change of FD and I_{FD10} from pH 5 to pH 7 in vivo. (c) Representative pseudocolour merged images of I_{FD10} labeled coelomocytes in wild type hermaphrodites at indicated times. (Scale bar: 10 μm) (d) Histograms of 480/430 ratios of maturing endosomes, at 5 min (pink), 17 min (purple) and 3 hr (cyan). (n=25 endosomes) (**The in vivo fold change for FD is obtained using FD70 since the uptake characteristics of FD10 is so poor that in vivo pH measurements are not possible to make with FD10).

FIG. 11 shows uptake properties of I_{FD10} by coelomocytes in C. elegans. Representative epifluorescence images of coelomocyte uptake (arrowheads indicate labeled coelomocytes) in a wild type adult hermaphrodite injected with (i) 3 μM solution of I_{FD10}, where [FD10]=11 μM (ii) 11 μM solution of free FD10, (iii) 3 μM solution of I_{FD10} co-injected with 30 μM solution of mBSA and (iv) 11 μM of FD10 co-injected with 30 μM solution of mBSA. Images are acquired under the same acquisition settings. (Scale bar: 100 μm).

FIG. 12 shows I_{FD10} is uptaken by surface ALBRs present on coelomocytes. Free (grey) and encapsulated (black) FD 10 are uptaken by different mechanisms in vivo. Uptake efficiency quantified by percentage of coelomocytes labeled post-injection with free and encapsulated FD10. All values are normalized relative to I_{FD10}. Also shown is the labeling efficiency of IFD10 and FD10 co-injected with 10 equiv. of mBSA. Error bars represent s.e.m. (n=20 worms).

FIG. 13 shows functionality of encapsulated FD10 is quantitatively retained in vitro and in vivo. (a) Representative pseudocolor images of 480/430 ratio of coelomocytes clamped at pH 5 and pH 7. (Scale bar: 10 μm) (b) The fold change in 480/430 emission ratio of I_{FD10} is preserved in vivo. Data presented here are the average of 2 independent readings. First, the ratio of emission obtained by exciting I_{FD10} at 480 nm and 430 nm is obtained at two extreme pH values. The value obtained at pH 7 (x) divided by the value obtained at pH 5 (y) is referred to as the fold change and is a quantitative measure of probe functionality.

FIG. 14 shows the size exclusion chromatogram of I_{FD10} complex. The black and gray bars indicate that chromatogram was followed at two different wavelengths—260 nm (black) for DNA absorbance and 488 nm (grey) for FITC in FD10 absorbance. The same data is present on right side which shows the ratio of DNA absorbance (red) and FITC absorbance (green).

FIG. 15 shows HPLC trace showing elution of only DNA icosahedron and not FD10 when DNA and FD10 are mixed in 1:4 ratio at 100 nM DNA concentration.

### DETAILED DESCRIPTION

The present disclosure relates to a method of delivering neutral bio-imaging molecule(s) to a cell, said method comprising act of encapsulating the neutral bioimaging molecule(s) within a DNA icosahedron and delivering the DNA icosahedron to the cell.

In an embodiment of the present disclosure, the encapsulating of the neutral bio-imaging molecule(s) within the DNA icosahedron comprises acts of:
a) assembling DNA molecules to obtain a semi-icosahedral DNA capsule; and
b) incubating the neutral bio-imaging molecule(s) with the semi-icosahedral DNA capsule, and ligating the semi-icosahedral DNA capsules to obtain neutral bio-imaging molecule(s)-DNA icosahedron complex, wherein the DNA icosahedron encapsulates the neutral bio-imaging molecule(s).

In another embodiment of the present disclosure, the neutral bio-imaging molecule is selected from a group comprising Fluorescent Dextrans preferably FITC Dextran and TMR Dextran, peptides, inorganic nanoparticles, fluorescent nanoparticles, magnetic nanoparticles, fluorescent proteins, PET imaging probes, radioactive probes, Raman active probes and functional proteins or any combination thereof.

In yet another embodiment of the present disclosure, the neutral bio-imaging molecule is at concentration ranging from about 0.5 mM to about 5 mM.

In still another embodiment of the present disclosure, the assembling is carried out by associating DNA junction selected from a group comprising V junction, U junction and L junction or a combination thereof to form semi-icosahedral DNA capsule.

In still another embodiment of the present disclosure, the incubating is carried out at pH ranging from about 6 to about 8, preferably about 7, at a temperature ranging from about 4° C. to about 55° C., preferably about 45° C., and for a time duration ranging from about 3 hours to about 5 hours, preferably about 4 hours.

In still another embodiment of the present disclosure, the DNA icosahedron encapsulating the neutral bioimaging molecule(s) is at concentration ranging from about 0.5 mM to about 5 mM and wherein the DNA icosahedron has pore size ranging from about 2 nm to about 3 nm, preferably about 2.8 nm.

In still another embodiment of the present disclosure, the ligating is carried out using chemicals selected from a group comprising N-Cyano Imidazole [NCI] and Cyanogen Bromide, preferably N-Cyano Imidazole [NCI]; at temperature ranging from about 15° C. to about 25° C., preferably about 20° C.

In still another embodiment of the present disclosure, re-incubation is carried out after the ligation for time duration ranging from about 24 hours to about 96 hours, preferably about 72 hours and at temperature ranging from about 0° C. to about 20° C., preferably about 4° C.

In still another embodiment of the present disclosure, the delivery is selected from a group comprising in-vivo, ex-vivo and in-vitro delivery.

In still another embodiment of the present disclosure, the in-vivo delivery is carried out by microinjecting the DNA icosahedron encapsulating the neutral bioimaging molecule(s); and wherein ex-vivo and in-vitro delivery is carried out by electroporation or pulsing the cell with the DNA icosahedron encapsulating the neutral bioimaging molecule(s).

In another embodiment of the present disclosure, pulsing the cells means adding the probe solution to the cells which are adhered on the imaging dish. The cells are adhered on glass slide. A drop (about 100 μL) of the probe (or labeling solution) is added to the cells, incubated for about 5 min to about 30 min. The cells are then washed with imaging buffer such as M1 buffer. Microinjection is done by loading the sample in a borosilicate glass capillary and injected into the organism immobilized on an agar pad.

In still another embodiment of the present disclosure, after the delivery, the DNA icosahedron encapsulating the neutral bioimaging molecule(s) is taken up by the cell through interaction of the cell receptors with the DNA icosahedron by pathways selected from a group comprising receptor mediated endocytosis, anionic ligand-binding receptor (ALBR) pathway, recycling pathways and fluid phase uptake pathway, preferably ALBR pathway.

The present disclosure also relates to a complex comprising DNA icosahedron encapsulating neutral bioimaging molecule(s).

In an embodiment of the present disclosure, the complex delivers the neutral bio-imaging molecule(s) to a cell.

In another embodiment of the present disclosure, the delivery is selected from a group comprising in-vivo, ex-vivo and in-vitro delivery

In yet another embodiment of the present disclosure, the in-vivo delivery is carried out by microinjecting the DNA icosahedron encapsulating the neutral bioimaging molecule(s); and wherein ex-vivo and in-vitro delivery is carried out by pulsing the cell with the DNA icosahedron encapsulating the neutral bioimaging molecule(s).

In still another embodiment of the present disclosure, the neutral bio-imaging molecule is selected from a group comprising Fluorescent Dextrans preferably FITC Dextran and TMR Dextran, peptides, inorganic nanoparticles, fluorescent nanoparticles, magnetic nanoparticles, fluorescent proteins, PET imaging probes, radioactive probes, Raman active probes and functional proteins or any combination thereof.

In still another embodiment of the present disclosure, the neutral bio-imaging molecule is at concentration ranging from about 0.5 mM to about 5 mM.

In still another embodiment of the present disclosure, the neutral bio-imaging molecule is a polymer based fluorescent molecule, functioning as a pH reporter devoid of any molecular recognition within the DNA icosahedrons.

In still another embodiment of the present disclosure, the DNA icosahedron encapsulating the neutral bioimaging molecule(s) is at concentration ranging from about 0.1 μM to about 3 μM and wherein the DNA icosahedron has pore size ranging from about 2 nm to about 3 nm, preferably about 2.8 nm.

The present disclosure also relates to a process for synthesising a complex comprising DNA icosahedron encapsulating neutral bioimaging molecule(s), said process comprising acts of:
a) assembling DNA molecules to obtain a semi-icosahedral DNA capsule; and
b) incubating the neutral bioimaging molecule(s) with the semi-icosahedral DNA capsule, and ligating the semi-icosahedral DNA capsules to obtain neutral bioimaging molecule(s)-DNA icosahedron complex, wherein the DNA icosahedron encapsulates the neutral bio-imaging molecule(s).

The present disclosure shows the entrapment of neutral bioimaging molecules like FITC dextran within the cavity of a DNA polyhedron without any molecular recognition or chemical conjugation between host (DNA icosahedron) and cargo (like FITC Dextran). This DNA polyhedron is structurally well defined and shows high encapsulation efficiency. Further, the endocytic properties of the DNA scaffold are imparted to its molecular cargo both in cellulo and in vivo.

In one aspect of the present disclosure, DNA capsules are used to encapsulate neutral bio-imaging agents. Neutral bio-imaging agents are materials that allow the DNA particles to be visualized after exposure to a cell or tissue. Bio-imaging includes imaging for the naked eye, as well as imaging that requires detecting with instruments or detecting information not normally visible to the eye, and includes imaging that requires detecting of photons, sound or other energy quanta Further, bioimaging agents should provide high signal to noise ratios so that they are detected in small quantities, whether directly, or by effective amplification techniques that increase the signal associated with a particular target. Examples include neutral stains, vital dyes, fluorescent markers, radioactive markers, enzymes or plasmid constructs encoding markers or enzymes.

The term DNA-Icosahedron of the present disclosure includes or encompasses other technically similar terms known to the person skilled in the art, such as DNA cage, DNA capsule, DNA polyhedra etc.

Many neutral imaging agents are loaded into a particle having a delivering molecule. In contrast, only a single neutral imaging agent linked to a delivering molecule is taken up by the same event. Since the internalization, intracellular transport, and recycling of cell surface receptors often requires significant turnaround time, the resultant direct uptake of signal molecules by a cell is slower than the uptake of signal molecules with a DNA capsule.

X-Ray contrast agents are also incorporated into DNA particles, which are delivered to a patient, tissue, or cell. X-ray contrast agents already known in the art include a number of halogenated derivatives, especially iodinated derivatives, of 5-amino-isophthalic acid.

Steps required for validation of the present disclosure would essentially involve:
Alteration of target specificity of DNA polyhedra to various tissues in organism like neurons or muscles.
Encapsulation of functional neutral imaging molecules for bioimaging inside the DNA cages.
Method of delivering neutral bio-imaging agents encapsulated in the host DNA icosahedron to cells.
Alteration of uptake properties of the encapsulated cargo by DNA icosahedron in cellulo and in vivo.
Better delivering of functional neutral bioimaging molecules like FD10—in terms of interactions and delivering.

The present method is technically superior to scaffolds of the prior art because (i) it is not limited to molecules that need to undergo molecular recognition with the host scaffold. This affords the following advantages: (i) Larger varieties of molecules may be encapsulated provided they have a size compatibility with the polyhedron. (ii) The size of the polyhedron is easily altered to encapsulate differently sized molecules. (iii) Guest molecules do not need to undergo a chemical reaction for encapsulation.

In contrast to the prior art, the present disclosure shows that DNA icosahedron encapsulates freely floating neutral bioimaging molecules (such as FITC dextran) within its cavity without any molecular recognition or chemical conjugation between DNA or FITC dextran. Further, the preservation of cargo functionality post-encapsulation in vitro and in vivo is shown. Since icosahedron is the most complex Platonic solid, it is shown to have maximum encapsulation volume while preserving the minimum pore size. Thus, one can efficiently encapsulate enough number of molecules within them while minimizing leakage of encapsulated molecules through its small pores.

The benefits of using DNA as a host molecule is that the stoichiometry and structure of DNA architectures can be programmed and controlled very precisely thus leading to uniform and homogeneous particles.

Another advantage of using DNA icosahedrons to encapsulate labelled neutral units, is that its assembly takes place in a programmed and controlled manner. Hence, it allows for controlling (a) degree of labelling, (b) specific positions for labelling the delivering moieties.

The advantage of a synthetic host based on a biological material such as DNA is that they are used to construct host-cargo complexes whose functionality is demonstrated in living systems. Further, a molecular recognition-free route to achieve this enables a strategy that is generalizable to many types of functional cargo. Importantly the functionality of the cargo post encapsulation is quantitatively demonstrable in vivo. In the present disclosure neutral bioimaging molecules such as FITC-Dextran (FD) is chosen as a cargo, since it satisfies the aforementioned criteria and possesses the following two properties. Firstly, the fluorescein moieties (FITC) on FD are pH sensitive, conferring on it the property to measure organellar pH inside living cells and whole organisms. Secondly, this neutral biopolymer does not interact with membrane-bound receptors and this confers on it the property to mark the fluid phase endocytic pathway. Importantly, non-functionalized FITC Dextran (FD) cannot be delivered to specific endocytic pathways due to this non-interacting property. Thus, any emergent properties of the cargo (FD) upon encapsulation in DNA polyhedra are measurable in terms of altered endocytic properties of the resultant DNA host-cargo complex. Simultaneously, cargo functionality post-encapsulation within a living system is quantitatively evaluated in terms of the other property, i.e., pH mapping ability. The present disclosure presents study on a) whether neutral bioimaging molecules such as about 10 kDa FITC-Dextran (FD10) could be encapsulated inside a synthetic, icosahedral DNA host; b) delivering the encapsulated cargo in cellulo and in vivo; c) whether the resultant host-cargo complex show any emergent behavior reflected in entirely new endocytic routes of uptake for FD10 and d) whether the functionality of the cargo in terms of pH sensing properties is preserved along with any manifested emergent property within a living organism.

The present disclosure reports the formation of a synthetic icosahedral DNA host-cargo complex and demonstrates the retention of the functionality of the encapsulated cargo along with emergent behavior within a living organism. The nature of association of the encapsulated cargo with the host scaffold within the host-cargo complex is probed and it is determined that the cargo shows minimal interaction with the host scaffold. Emergent properties of the host-cargo complex in cellulo are manifested in dramatic alteration of uptake pathways of encapsulated FD10 versus free FD10 in Drosophila hemocytes. Free FD10 is uptaken by the fluid phase pathway whereas encapsulated FD10 is uptaken exclusively by the anionic ligand-binding receptor (ALBR) pathway indicating that encapsulated FD10 is now targeted to ALBRs. This is also borne out in vivo, where the host-cargo complex is uptaken only by specific cells (coelomocytes) in C. elegans that expressed ALBRs. The functionality of the encapsulated cargo in vivo is quantitatively demonstrated by mapping pH changes associated with endosomal maturation along the ALBR pathway in coelomocytes of C elegans.

Complex Formation Between DNA Icosahedron and FD10

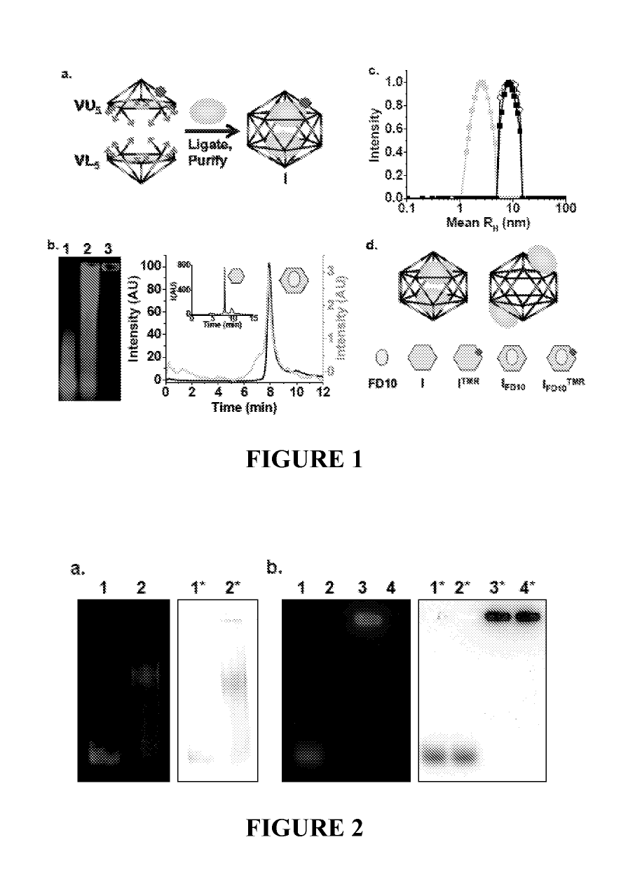

DNA icosahedra are assembled using the modular assembly protocol which involves the association of two types of 5 way junctions V and U (or V and L) to first form half icosahedra VU₅ (or VL₅) followed by the further assembly of 1:1 VU₅:VL₅ (FIG. 1a and FIG. 2) into icosahedral DNA capsules, I, in near quantitative yields. A five way junction is a star like structure formed when five single stranded DNA oligonucleotides hybridize together. This junction represents a single vertex of the DNA icosahedron. The abbreviations V, U and L are just given to indicate their position along the icosahedron. In order to encapsulate the cargo, a neutral bioimaging molecule (such as FD10), inside these icosahedral capsules, the two halves VU₅ and VL₅ are incubated in 1:1 ratio in presence of 2 mM FD10, such that there is at least one FD10 molecule per 1000 nm³ which is the measured minimum encapsulable volume of the DNA icosahedron (FIG. 1a). Upon ligation, the excess free FD10 is separated from DNA icosahedra by gel electrophoresis followed by size exclusion chromatography (SEC-HPLC) (FIG. 1b). This yielded DNA icosahedra that showed fluorescence corresponding to FD10, despite rigorous purification from free FD10, suggesting the formation of an I_{FD10} complex. The ratio of absorbance at 254 nm to 488 nm of the eluted fraction corresponding to the I_{FD10} complex gave a stoichiometry of FD10:I of 2:1 indicating an average of two FD10 molecules associated per DNA cage (FIGS. 1b, 1d, 14). The DNA is recovered in quantitative yield i.e. more than 75% DNA icosahedron. This concentration of DNA carries less than 1% of the fluorophore FD10 used initially for encapsulation i.e. 2 mM (FIG. 14).

The concentration of FD10 to be used depends on how many molecules need to be present in the encapsulable volume of icosahedron. So the workable range of concentration of FD10 to be used will vary from 0.2 mM to 20 mM which will cause 0.1 to 10 molecules of FD10 per 1000 nm³. All the DNA concentrations used i.e. VU5, VL5 are 3.33 μM, FD10=2 mM, phosphate buffer (Sodium dihydrogen phosphate and Disodium hydrogen n phosphate)=10 mM+100 mM NaCl+1 mM MgCl₂ (Magnesium chloride), pH 7, NCI=50 mM.

In order to determine the nature of association of FD10 with the DNA icosahedron-I, the I_{FD10} complex is characterized by dynamic light scattering (DLS). DLS of a sample of pure FD10 showed peaks corresponding to an R_{H} of 2.6±1.0 nm (FIG. 1c). DLS of a sample of DNA icosahedra I, showed an R_{H} of 9.2±0.1 nm. DLS of a pure sample of I_{FD10} complex showed an R_{H} of 9.3±0.4 nm, consistent with the measured dimensions of the DNA icosahedron. There are no peaks at R_{H} ˜3 nm corresponding to free FD10 (FIG. 1c).

Labeled and unlabeled oligonucleotides are obtained from IBA-GmBh (Germany) and Bioserve (India), respectively. FITC dextrans, and nigericin are purchased from Sigma (USA); FD10 is obtained from Invitrogen (USA); TD10 is TMR dextran, 10 kDa purchased from Invitrogen. mBSA and N-cyano imidazole (NCI) are synthesized in-house, by following the procedure mentioned below:
a) Procedure for making mBSA: 20 mg of Bovine serum albumin (BSA) is dialyzed in 0.1M sodium carbonate bicarbonate buffer, pH 9.0 for one hour at room temperature. 50 mg of maleic anhydride is added to the dialyzed protein solution (5 mg/mL), while adding solid sodium carbonate to maintain pH. (Optimal pH for malylation is 9) pH during the reaction is monitored using pH strips. The reaction proceeded with release of CO2. End of reaction is gauged by drop in effervescence that indicates complete use of available of maleic anhydride. Reaction mixture is left standing at room temperature for 1 hour prior to dialysis against phosphate buffer saline (PBS), pH 8.0 for 24 h at 4° C. mBSA thus made is stored at −20° C.
b) Procedure for making NCI: A solution of 5.5 g (0.03 mole) Cynaogen bromide (BrCN) in 25 mL benzene is added dropwise with stirring to a solution of 3.2 g (0.05 mole) imidazole in benzene. The mixture is warmed to 50° C. After addition the mixture is stirred at 50° C. for an additional 5 min, then cooled to 4° C. for overnight. Next day, a yellow solid of hydrogen bromide (HBr) will precipitate and the supernatant solution is filtered through Whatman filter paper. A small additional wash of benzene is given. The filtrate is concentrated to dryness for long time using rotavapour. The remaining white crystalline solid (NCI) is removed and distributed in small quantities in eppendorfs and stored at −20° C. It can be stored for at least 2 weeks at −20° C. and its decomposition is indicated by the colour change from white to yellow.

Methods for Construction and Characterization of DNA Icosahedron is Provided Below:

DNA icosahedra are constructed from three distinct five way junction (5WJ) components V, U and L, with programmable overhangs. Each 5WJ module V, U and L are constructed from equimolar ratios of the respective five phosphorylated single strands. At 20 μM, V forms a complex with L in a 1:5 ratio. The complementary module VU₅ is similarly synthesized from components V and U. At this stage, contiguously hybridized strands in VU₅ and VL₅ are chemically ligated with N-Cyano imidazole (NCI), to enhance stability. The two different modular assemblies, VU₅ and VL₅, with ten identical overhangs each, that are complementary to each other, form a complex with each other in a 1:1 ratio and the contiguous termini are ligated again with NCI to yield a complex I (icosahedron).

FD10 is Encapsulated within DNA Icosahedra

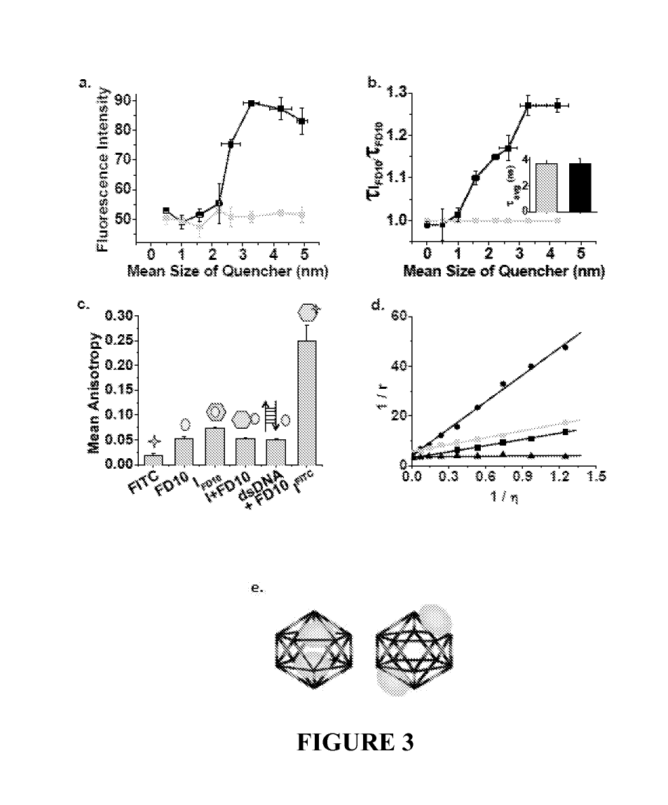

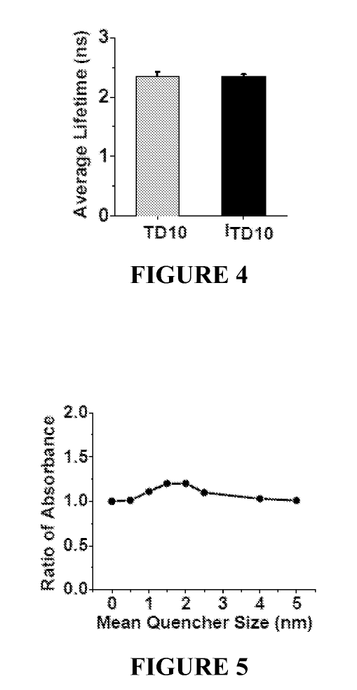

The I_{FD10} complex could have the FD10 externally associated, or internally entrapped within the DNA Icosahedron (FIG. 1d). The former would show an altered hydrodynamic radius by DLS, which is not observed. In order to confirm the latter model, I_{FD10} complex (I_{FD10}) is subjected to quenchers of different sizes ranging from 0.5-5 nm diameter and their abilities to quench the fluorescence of the fluorescein moiety by collisional quenching studied. In the absence of any added quencher, fluorescence lifetime of DNA-associated FD10 (I_{FD10}) is the same as free FD10 (Inset, FIG. 3b) indicating that the fluorophore in I_{FD10} is unaffected by the DNA scaffold I (FIG. 4). Next, the fluorescence intensity is monitored by the addition of quenchers of different sizes to a solution of I_{FD10}. If the FD10 is encapsulated within the DNA icosahedra, only quenchers smaller than the measured pore size will be able to access the interior of the polyhedron and quench the fluorescence, while quenchers larger than the pore size will not. I_{FD10} and FD10 (50 nM) are treated with quenchers of various sizes such as iodide (0.35 nm), Amino TEMPO (1 nm), Nanogold (1.5 nm) and gold nanoparticles (GNPs) of sizes 2, 3, 4 and 5 nm respectively. Each species of quencher has an intrinsically different ability to collisionally quench fluorescence and this is corrected for by using that concentration of the quencher which results in a 50% decrease in fluorescence intensity of the sample. This is obtained from the reciprocal of their measured Stern Volmer constants (K_{SV}, Table 1 [below] and FIG. 5). When FD10 and I_{FD10} (50 nM each) are subjected to 1/K_{SV} concentration of each quencher, it is observed that in the case of FD10, quenchers of all sizes quenched the fluorescence intensity by 50% (FIG. 3a). However, in the case of I_{FD10}, only quenchers below ˜2.2 nm diameter could quench the fluorescence by 50%. Quenchers of sizes≧3 nm are ineffective at quenching fluorescence, while quenchers between 2.2-3 nm diameters could only partially quench fluorescence (FIG. 3a). This is further confirmed by an analogous study of fluorescence lifetimes of the fluorescein moieties present on free FD10 and I_{FD10}) (FIG. 3b). Fluorophore lifetime is a direct reporter of the quenching environment of the fluorophore and the mechanism of fluorescence quenching by different quenchers. Fluorescein lifetime in I_{FD10} and free FD10 showed comparable decrease for small sized quenchers, while quenchers larger than 3 nm diameter could not decrease the lifetime for I_{FD10} (FIG. 3b).

TABLE 1
Stern-Volmer constants for various quenchers for FD10
Characterization of all quenchers for their quenching abilities by measuring their Stern-
Volmer constants with respect to FD10.

| Quencher | Size | K_{SV} [M⁻¹] | K_{D} [M⁻¹] | K_{S} [M⁻¹] | K_{O} [M⁻¹s⁻¹] | Conc. [M] |
|---|---|---|---|---|---|---|
| Iodide | 0.5 nm | 6.1 ± 0.12 | 4.91 ± 0.17 | 0.41 ± 0.09 | 1.23 × 10⁹ | 0.164 |
| TEMPO | 1 nm | 195 ± 0.046 | 95 ± 0.01 | 40 ± 0.004 | 48.7 × 10⁹ | 0.005 |
| TEMPO-H | 1 nm | 27.6 ± 0.002 | 52 ± 0.003 | −0.01 ± 0.002 | 6.9 × 10⁹ | 0.036 |
| TEMPO-A | 1 nm | 21.3 ± 0.003 | 11.6 | 9.7 | 5.3 × 10⁹ | 0.05 |
| Nanogold | 1.5 nm | 2.4 × 10⁴ ± 0.002 | 1.8 × 10³ ± | 250 ± 300 | 4 × 10⁴ | 41.6 × 10⁻⁶ |
| | | | 400 | | | |
| Gold nano | 2.5 | 14.2 × 10⁶ ± | 85 × 10⁶ ± | −0.04 ± 0.008 | 3.55 × 10³ | 70 × 10⁻⁹ |
| particles | onwards | 0.003 | 10 × 10⁸ | | | |
| | (5 nm) | | | | | |

K_{SV} = Stern-Volmer constant;
K_{D} = constant for dynamic quenching;
K_{S} = constant for static Quenching;
K_{q} = quenching rate constant.

Lifetime Measurements

Lifetime measurements are performed at 5 μM fluorophore concentration using a frequency domain Fluorolog Tau 3 (Horiba Jobin Yvon, Japan). The S and T channels are calibrated using glycogen as a standard and the operating frequency is 10 MHz. For each sample, the frequency and modulation spanned from 10 MHz to 150 MHz using 7-10 intermediate frequency readings. The data obtained is fitted using the associated software and readings showing χ² value less than 1.2 are selected.

Quencher Characterization

Quenchers of different sizes are selected based on literature reports; these included Iodide (0.5 nm), Amino TEMPO (1 nm) and Nanogold (1.5 nm). Quenchers in the regime 2 nm-5 nm are all gold nanoparticles and are synthesized using previously reported methods and characterized by TEM. The quencher of size 2.5 nm is 1 kDa dextran (size=2.4-2.6 nm diameter by DLS) that is coupled to carboxy TEMPO using dicyclohexylcarbodiimide and purified by SEC-HPLC.

Factors affecting the encapsulation of neutral bioimaging molecules within the host DNA icosahedrons are:
(i) Concentration: Neutral molecules such as fluorescent dextrans like FD10, TD10 and fluorescent proteins like GFP, RFP, etc require high concentrations whereas the charged molecules can be encapsulated at low concentrations depending on the interactions. The suitable concentration ratio to encapsulate neutral bioimaging molecules within DNA icosahedra ranges from about 500 to about 5000. The bioimaging agents which have charge can also be encapsulated within DNA icosahedron. However, for such agents the concentration ratios need to be standardized and calculated.
(ii) Charge of the cargo: Charged molecules are encapsulated at low concentrations depending on the interactions. Thus, since the DNA host is negatively charged, positively charged molecules are encapsulated within it at low concentrations due to the host-cargo electrostatic interaction. There is a disadvantage in using positively charged bioimaging cargo, since it will stick non specifically on the negatively charged DNA backbone. Negatively charged cargo, are encapsulated them using buffers with positive ions like Mg²⁺. These are encapsulated within DNA icosahedron using a combination of DNA icosahedron and cargo in the ratios of 1:4 to 1:20. Usually, it is very difficult to synthesize negatively charged cargoes like GNPs and QDs beyond the concentrations of 20 μM, since they tend to aggregate at high concentrations.
(iii) Shape: Another factor which controls the uptake property of the cargo by host DNA is the shape of the cargo. Cargo molecules with rigid structure are accommodated in high numbers within the DNA. On the other hand, molecules like FD10 or fluorescent proteins are flexible polymers and are not rigid. Rigid molecules have fixed surface area and surface charge display using which, they interact with DNA molecules in a definite and enhanced manner. However, such electrostatic interaction and uniform distribution of charges are lost for flexible molecules. Hence for flexible molecules it becomes difficult to predict the concentrations suitable to see encapsulation. Thus, flexible molecules require higher concentrations to observe encapsulation, whereas for rigid molecules, lower concentrations is sufficient to observe encapsulation. A concentration of about 2 mM to about 5 mM for a neutral molecule which does not interact with DNA is found to be suitable (this observation has been supported experimentally by anisotropy results).
(iv) Molecular interactions between the host and cargo: Interaction between the host and cargo play an important role to control the number of molecules that are encapsulated within DNA icosahedron. Neutral bioimaging molecules being neutral do not interact with the negatively charged DNA icosahedron. So the encapsulation of the neutral bioimaging molecules is due to volume overlap between icosahedron and FD10. Hence it is seen that only 2 FD10 molecules are encapsulated within each icosahedron.

The present disclosure is further elaborated with the help of following examples and associated figures. However, these examples should not be construed to limit the scope of the present disclosure.

### EXAMPLES

### Example 1

### Encapsulation of Molecular Cargo within DNA Icosahedron

The two half icosahedra VU₅ and VL₅ (3.33 μM each) are mixed in presence of 2 mM of the desired cargo i.e., FD (FD4, 10, 20, 40, 70, 150) in 10 mM phosphate buffer, pH 6, incubated at 45° C. for 4 h and annealed to RT. After incubation for 72 h at 4° C., it is chemically ligated using NCI at RT and again incubated for 72 h at 4° C. Thereafter, the solution containing DNA host (I) and cargo (FD10) is run on 0.8% agarose gel in 1×TAE buffer. The band corresponding to I is excised and eluted in 100 mM NaCl and 1 mM MgCl₂. The eluted solution is further purified by either size exclusion chromatography (BioSep S3000, Phenomenex) on a Shimadzu HPLC system or dialysis using a 50 kDa MWCO CelluSep membrane (MFPI, USA) to remove trace amounts of free cargo (FD10) from the DNA host-cargo complex (I_{FD10}). Please refer to Table 2.

Encapsulation Ability of Icosahedron as a Function of Cargo Size

TABLE 2
Encapsulation efficiency of DNA icosahedron as a function of cargo size

| | | Measured^{b} | No: of | | | |
|---|---|---|---|---|---|---|
| Cargo^{a} | Manufacturer | diameter ± | FITCs | Total^{d} 254 | Total^{d} 488 | |
| size | reported | polydispersity | per | absorbance | Absorbance | Ratio (Cargo |
| (kD) | diameter (nm) | (nm) | cargo^{c} | (AU) | (AU) | per host) |
| 4 | 2.8 | 2.72 ± 1.04 | 0.4 | 755157 | — | undetectable |
| 10 | 4.6 | 5.24 ± 1.72 | 2.5 | 4716205 | 178324 | 2.35 |
| 20 | 6.6 | 6.12 ± 1.94 | 3 | 684470 | 80081 | 2.11 |
| 40 | 9.0 | 8.24 ± 2.56 | 4.4 | 31435136 | 2045026 | 0.33 |
| 70 | 12.0 | 10.86 ± 3.62 | 7.7 | 12402272 | 140840 | 0.3 |
| 150 | 17.0 | 11.24 ± 5.14 | 16.6 | 3123181 | 79957 | 0.01 |

^{a}All cargo were FITC-Dextrans of indicated size/molecular weight;
^{b}Experimentally measured by dynamic light scattering of a 1 mM sample;
^{c}The average loading as specified by the manufacturer;
^{d}Area under the SEC-HPLC peak monitored at 254 nm and 488 nm;
^{e}Encapsulation efficiency represented by the ratio of cargo molecules present within the fraction corresponding to the DNA host post encapsulation, ligation and purification from excess cargo.

### Example 2

### DLS Experiments

DLS studies are performed on a DynaPro-99 unit (Protein Solutions) at 25° C. Buffer and samples (FD10, I_{FD10}, I obtained from the above Experiment) are filtered using 0.02 μm filters (Whatman, England) and 0.22 μM filters (Millipore), respectively and spun at 10000 rpm for 10 min prior to use. The buffer used is: Phosphate buffer, 10 mM+Sodium chloride, 100 mM+Magnesium chloride, 1 mM, pH 7. The experimental settings used are: acquisition time, 3 s; S/N threshold, 2.5; and sensitivity, 70%. The sample is illuminated with an 829.4 nm laser; scattering intensity at 90° and its autocorrelation function are measured simultaneously. Fluctuations greater than 15% in the scattering intensity are excluded from the analysis. The DynaLS software (Protein Solutions) is used to resolve acquisitions into well-defined Gaussian distributions of hydrodynamic radii. The size of FD10 is measured at 1 mM concentration while those of I_{FD10} and I are measured at 1 μM concentration of host. (FIG. 1c).

DLS of a sample of pure FD10 showed peaks corresponding to an R_{H} of 2.6±1.0 nm (FIG. 1c). DLS of a sample of DNA icosahedra I, showed an R_{H} of 9.2±0.1 nm. DLS of a pure sample of I_{FD10} complex showed an R_{H} of 9.3±0.4 nm, consistent with the measured dimensions of the DNA icosahedron. There are no peaks at R_{H} ˜3 nm corresponding to free FD10 (FIG. 1c).

The DLS results obtained show that the icosahedron can be formed in correct dimensions even in presence of 2 mM of FD10. Further, it shows that association of FD10 with icosahedron does not change the dimensions of I_{FD10} complex, thus implying that FD10 might be present inside the DNA icosahedron rather than sticking on it from outside.

### Example 3

### Fluorescence Intensity and Lifetime Measurements

For intensity based quenching, the fluorescein concentration in FD10 and I_{FD10} is maintained at 50 nM in phosphate buffer of pH 7. All the quenchers are characterized from their measured Stern-Volmer plots for FD10. These concentrations of quenchers are added to FD10 and I_{FD10}. After addition of quencher, the solution is equilibrated for 2 min and fluorescence intensities measured. The percentage of fluorescence remaining is measured and plotted as percentage fluorescence. All readings are corrected for dilution. For lifetime measurements, the concentration of fluorescein in FD10 and I_{FD10} is maintained at 5 μM and quencher concentrations are increased accordingly. The lifetimes presented are average lifetimes in nanoseconds calculated from two component fitting of lifetimes using the formula:

<τ_{avg}>=(τ₁f₁²+τ₂f₂²)/(τ₁f₁+τ₂f₂)

where τ₁ and τ₂ are the lifetimes of two components and f₁ and f₂ are the respective fractions of the component.

If the FD10 is encapsulated within the DNA icosahedra, only quenchers smaller than the measured pore size will be able to access the interior of the polyhedron and quench the fluorescence, while quenchers larger than the pore size will not. I_{FD10} and FD10 (50 nM) are treated with quenchers of various sizes such as iodide (0.35 nm), Amino TEMPO (1 nm), Nanogold (1.5 nm) and gold nanoparticles (GNPs) of sizes 2, 3, 4 and 5 nm respectively. Each species of quencher has an intrinsically different ability to collisionally quench fluorescence and this is corrected for by using that concentration of the quencher which results in a 50% decrease in fluorescence intensity of the sample. This is obtained from the reciprocal of their measured Stern Volmer constants (K_{SV}, Table 1 [below] and FIG. 5). When FD10 and I_{FD10} (50 nM each) are subjected to 1/K_{SV} concentration of each quencher, it is observed that in the case of FD10, quenchers of all sizes quenched the fluorescence intensity by 50% (FIG. 3a). However, in the case of I_{FD10}, only quenchers below ˜2.2 nm diameter could quench the fluorescence by 50%. Quenchers of sizes 3 nm are ineffective at quenching fluorescence, while quenchers between 2.2-3 nm diameters could only partially quench fluorescence (FIG. 3a). This is further confirmed by an analogous study of fluorescence lifetimes of the fluorescein moieties present on free FD10 and I_{FD10} (FIG. 3b). Fluorophore lifetime is a direct reporter of the quenching environment of the fluorophore and the mechanism of fluorescence quenching by different quenchers. Fluorescein lifetime in I_{FD10} and free FD10 showed comparable decrease for small sized quenchers, while quenchers larger than 3 nm diameter could not decrease the lifetime for I_{FD10} (FIG. 3b).

Thus the encapsulation of FD10 within DNA icosahedron is done in bulk using quenchers of different sizes. Since FD10 has fluorescence property, using quenching studies, it is proved that encapsulation is done in bulk, i.e. most of the empty DNA icosahedra heads get encapsulated with the cargo FD10 via the instant method of encapsulation.

Further, it is known that there are two possibilities for entrapment of neutral bioimaging molecules inside a DNA capsule. The cargo is either encapsulated inside the DNA capsule or it is attached on the outer surface of the DNA capsule (FIG. 3e). Both these possibilities are tested by subjecting FITC dextran loaded icosahedra to quenchers of various sizes. If the cargo is attached on the outer surface of the DNA capsule, it should be quenched equally by all the quenchers. However, if the cargo is encapsulated inside the DNA capsule, it is quenched only by those quenchers that are smaller than the pore size of the DNA capsule. In the instant invention, it is found that only quenchers smaller than 3 nm (which is the pore size of the instant DNA capsule) are able to quench the FITC dextran associated with DNA icosahedron and quenchers of size larger than 3 nm are unable to quench the FITC dextran (FIGS. 3a and 3b). This indicates that FITC dextran is encapsulated within the DNA capsule in the instant disclosure.

### Example 4

### Mobility of Entrapped Cargo within I_{FD10}

To further probe the nature of association of the FD10 cargo with its synthetic host I, fluorescence anisotropy of fluorescein moieties present on entrapped FD10 is monitored (Table 3). This reports on segmental motion as well as global rotation corresponding to dextran tumbling. Both rotations cause fluorescence depolarization, which is reflected in fluorescence anisotropy.

Anisotropy and Perrin Plot Measurements

Anisotropy experiments are performed using 50 nM of fluorophore per sample, on Fluorolog 3 L instrument (Horiba Jobin Yvon, Japan), where the polarizing angles are fixed (90°). Before each experiment, the g factor is calibrated using FITC as a standard.

For every sample, time series of anisotropy is acquired, and the mean of each time series is selected. For Perrin plot, the samples are prepared in 0, 10, 20, 30, 40, 50, 60, 70, 80 and 90% glycerol (w/v), maintaining the fluorophore concentration at 50 nM.

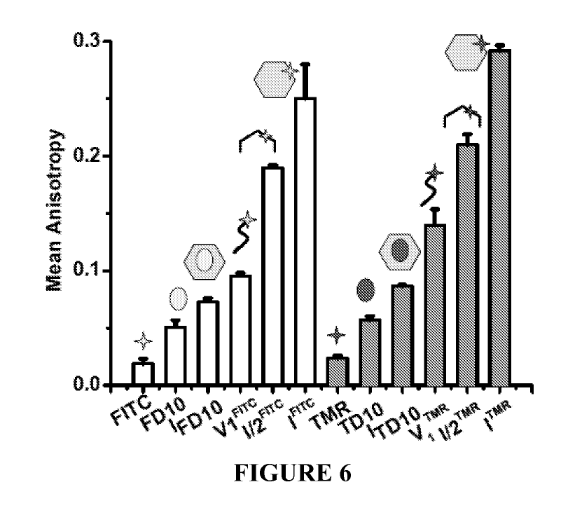

As shown in FIG. 3c, the fluorescein moieties of FD10 showed anisotropy of 0.05±0.01, which suggests that in FD10, the major component of the anisotropy of fluorescein comes from the free segmental rotation as reflected in the low anisotropy values (FIG. 6). Interestingly, in the I_{FD10} complex, fluorescein anisotropy is only marginally higher, 0.073±0.002, indicating that its motion is not affected upon complexation with the DNA icosahedron. A 2:1 mixture of externally added FD10 to preformed I showed anisotropy consistent with free FD10. This is further confirmed by anisotropy of a mixture of FD10 and linear, duplex DNA in the stoichiometry of the host—cargo complex, I_{FD10}. In order to check the extent of anisotropy increase when a fluorophore is actually attached to the DNA host, anisotropies between I carrying a single FITC (I^{FITC}) and the I_{FD10} complex are compared. The latter showed anisotropy of 0.073±0.003. I^{FITC} is made by incorporating a FITC tag on one of the component oligonucleotides V₁^{FITC} of the 5WJ ‘V’ of the half icosahedron VU₅. The anisotropy values of the fluorophore reflect its rotation while attached to the DNA structure as it changes from V₁^{FITC} to I/2^{FITC} to I^{FITC} (FIG. 3c and FIG. 6). Thus I^{FITC} showed a much higher anisotropy of 0.25±0.03.

To further explore the association of FD10 with I in the host-cargo complex, I_{FD10}, rotational correlation times (θ) of fluorescein present on free FD10 and I_{FD10} are determined. These are obtained by measuring anisotropy (r) of a sample as a function of viscosity (η) of the medium. 50 nM of sample is added to solutions containing different proportions of glycerol in water that yielded solutions of desired viscosities. Anisotropies of these samples are plotted as a function of solution viscosity giving the Perrin plot for FITC, free FD10 and I_{FD10}, the slope of which gives the rotational correlation time (θ) of fluorescein in these samples (FIG. 3d and Table 4). Free FD10 showed θ=2.2 ns while that of I_{FD10} is 3.1 ns. Fluorescein covalently attached to the synthetic host in I^{FITC} showed θ=140 ns.

TABLE 3
Anisotropy values

| Molecular Species | Mean Anisotropy Value | Standard Deviation |
|---|---|---|
| FITC | 0.01898 | 0.00392 |
| FD10 | 0.05089 | 0.00612 |
| IFD10 | 0.07314 | 0.00272 |
| Icosa + FD10 | 0.05117 | 0.00407 |
| dsDNA + FD10 | 0.04886 | 0.00292 |

TABLE 4
Perrrin Plot values

| Molecular Species | Rotational Correlation Time (ns) |
|---|---|
| FITC | 0.3 |
| FD10 | 2.2 |
| I_{FD10} | 3.1 |
| I^{FITC} | 140 |

### Example 5

### I_{FD10} is Targeted to Anionic Ligand Binding Receptors

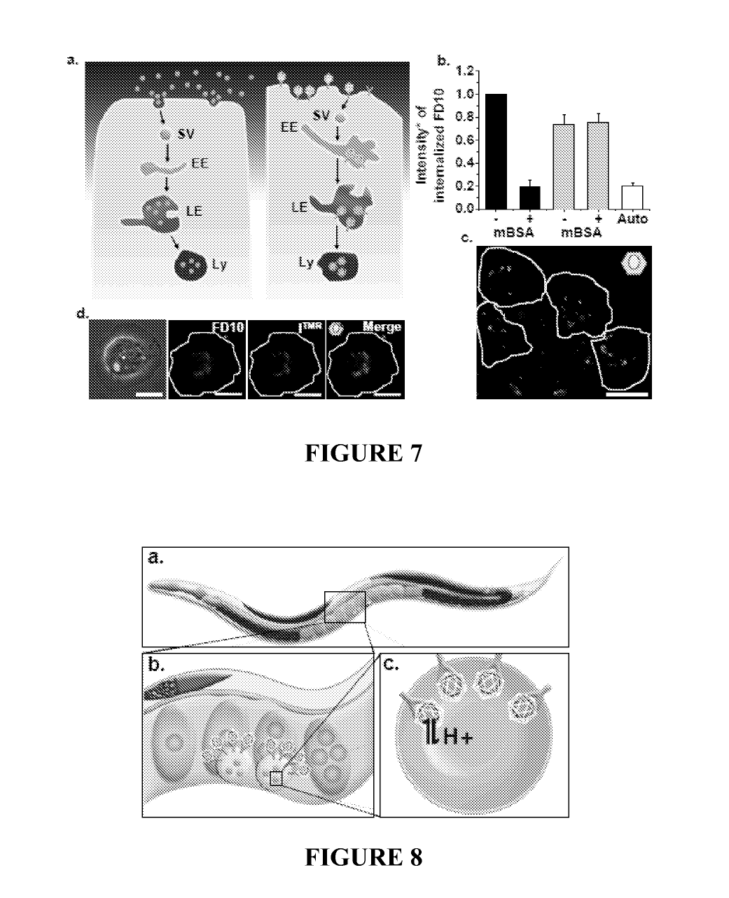

In order to study if the encapsulation of FD10 within a host-cargo complex manifests different properties from free FD10, the instant disclosure proceeded to explore uptake properties of free and host-encapsulated FD10 in cellulo. Drosophila hemocytes are a widely used model system to study the mechanisms of endocytosis of different types of molecules (FIG. 7a). When Drosophila hemocytes are pulsed with 12 μM free FD10, the latter is found to be localized in punctate structures. This uptake in cells is quantified in terms of whole cell intensity (FIG. 7b). Hemocytes are then pulsed with 12 μM I_{FD10} (DNA: 3 μM, FD10: 12 μM) for 5 min and chased for 5 min. Fluorescence images of these cells (FIG. 7c) showed that I_{FD10} is similarly localized in punctate structures, which showed slightly higher uptake than free FD10. When hemocytes are pulsed with 12 μM free FD10 in the presence of excess maleylated BSA (mBSA), its uptake by hemocytes is unaffected as expected for fluid phase endocytosis, and consistent with previous studies. Interestingly, when hemocytes are pulsed similarly with 12 μM I_{FD10} (DNA: 3 μM, FD10: 12 μM) and excess mBSA, uptake is completely abolished and intensities are commensurate with the level of autofluorescence in the cells (FIG. 7b). When Drosophila hemocytes are pulsed with a sample of TMR-labeled DNA icosahedra loaded with FD10 (I_{FD10}^{TMR}), chased and imaged, they showed colocalization (FIG. 7d) of the synthetic DNA host (in the TMR channel) and cargo FD10 (in the FITC channel). This confirms that when pulsed with I_{FD10}, the FD10 present in endosomes along this altered pathway is present along with its synthetic DNA host.

### Example 6

### Cell Culture and in Cellulo Endocytic Assays

Drosophila hemocytes are isolated in complete medium using previous methods. Prior to pulsing, the cells are washed using Medium 1 (150 mM NaCl, 5 mM KCl, 1 mM CaCl₂, 1 mM MgCl₂, 20 mM HEPES, pH 7.0, supplemented with 1 mg/mL BSA and 2 mg/mL glucose). After washing the cells with Medium 1, the labeling solution is added. The different labeling solutions used are FD10, I_{FD10} or I_{FD10-TMR}. For pulsing, FD10 (12 μM), I_{FD10} (DNA: 3 μM, FD10: 12 μM) or I_{FD10} TMR (DNA: 3 μM, FD10: 12 μM) in M1 buffer are added to these cells and incubated for 5 min. Labeling solution is then removed and replaced with M1 buffer for 5 min. After this, the cells are washed with M1 buffer and immediately imaged. Healthy cells are identified by the presence of well developed lamellipodia. The area of lamellipodia is used to demarcate the perimeter of cells and drawn as a white line in fluorescent images indicating the perimeters of respective cells.

M1 Buffer:

Sodium chloride, 150 mM

Potassium chloride, 5 mM

Calcium chloride, 1 mM

Magnesium chloride, 1 mM

HEPES (4-(2-hydroxyethyl)-1-piperazineethanesulfonic acid), 20 mM,

pH=7.0

For competition assays, hemocytes are divided into 5 plates. In the first plate, cells are untreated, and used to measure autofluorescence. In two plates, cells are pulsed with FD10 and I_{FD10}. In the remaining two plates, the surface receptors are saturated with 30 μM mBSA for 5 min. After this, the cells are pulsed with I_{FD10}/FD10 (DNA: 3 μM, FD10: 12 μM), containing 30 μM mBSA for 5 min, chased, washed and imaged. Experiments are performed in duplicate (FIG. 7).

The results obtained from the cellular uptake assay show that DNA icosahedron ferries the encapsulated FD10 by the ALBR pathway within the cells which has been observed by mBSA competition experiments. This is called altered uptake of the encapsulated cargo.

### Example 7

### Altered Endocytic of I_{FD10} is Manifested In Vivo

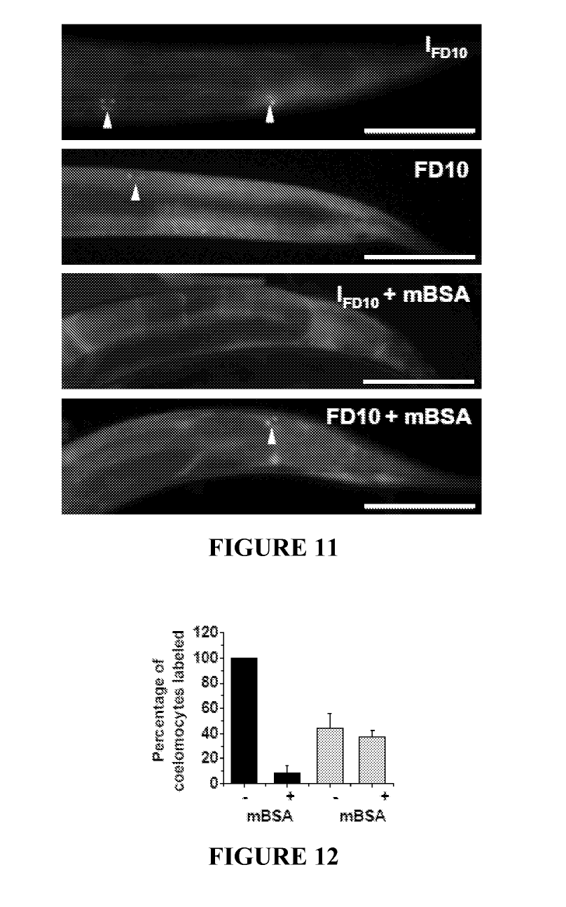

The instant disclosure presents a study on the uptake of encapsulated I_{FD10} by coelomocytes in C. elegans in comparison to free FD10. Free FD10 is a well known marker of fluid phase endocytosis in C. elegans characterized by poor coelomocyte uptake even at concentrations of 100 μM. This therefore serves as a useful comparator of uptake efficiency of FD10 when encapsulated inside DNA icosahedra. When a solution of 3 μM I_{FD10} containing 11 μM FD10, quantified by fluorescein absorbance, is microinjected into C. elegans, it marked 60±3% of coelomocytes as against 29±7% with 11 μM free FD10 (FIG. 12). Importantly, it is seen that uptake of I_{FD10} occurred only by coelomocytes. Representative images post-injection of comparable quantities of IFD10 and FD10 are shown in FIG. 11. Such cell-selective uptake indicates the involvement of receptors for the host-cargo complex in vivo. Given that DNA structures have been shown to undergo uptake by coelomocytes of C. elegans by receptor mediated endocytosis through scavenger receptors, a competition assay is carried out with mBSA in vivo. When a 3 μM solution of DNA icosahedra containing 10 μM FD10 is co-injected with 30 μM mBSA, the uptake of I_{FD10} is dramatically reduced to 4±3% due to competition, whereas the uptake of free FD 10 remained unchanged (22±3%) upon same treatment with mBSA (FIG. 12). This shows that FD10, which has otherwise weak uptake by coelomocytes, undergoes targeted uptake by coelomocytes due to the DNA casing of I_{FD10} via the ALBR or scavenger receptor pathway. This indicates that physical encapsulation of FD10 within the synthetic host DNA capsule changes biochemical interactions within a living organism at the level of the cargo (FD10). Thus the host is able to ferry the encapsulated cargo to only those cells that present the relevant receptor and uptake in those cells is driven by receptor mediated endocytosis. Upon co-injection with mBSA, the host-organism molecular interactions may be manipulated by saturating the relevant receptors with mBSA that acts as a competitor. In such a scenario, both cellular uptake and targeting properties are lost. The encapsulated cargo now behaves similarly to the free cargo indicating that knocking out molecular interactions between the synthetic host and organism also abolishes any emergent behavior of the host-cargo complex. This indicates that emergent behavior of the cargo in vivo is entirely due to encapsulation within the synthetic host.

### Example 8

### Altered Endocytic Uptake of I_{FD10} is Manifested In Vivo

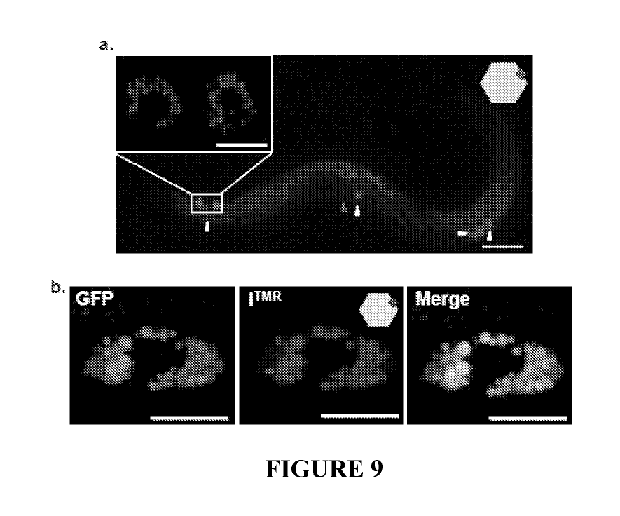

Given the stark alteration of molecular interactions and hence endocytic uptake pathways between free and encapsulated cargo in cellulo, studies are undertaken to see if the same is observed in vivo. The uptake of free FD10 and I_{FD10} inside C. elegans is assessed. C. elegans is a nematode that contains scavenger cells, called coelomocytes, which endocytose fluid from the pseudocoelom (FIGS. 8 and 9).

C. elegans Strains and In Vivo Endocytic Assays

Standard methods are used to maintain C. elegans. Wild type strain used is the C. elegans isolate from Bristol (N2); arIs37[pmyo-3::ssGFP] is used for colocalization of I^{TMR} and GFP. Microinjections are performed. Samples are injected in the dorsal side of pseudocoelom, opposite the vulva, of one day old hermaphrodites. Injected worms are transferred to NGM plates (+OP50), incubated at 22° C. for the stated time-points, and imaged. Colocalization of GFP and TMR is performed by injecting 3 μM of I^{TMR} into arIs37 worms. Uptake assays are performed by injecting 3 μM of I_{FD10} alone, and with 10 equivalents of mBSA, into N2 worms. The same is done with an equivalent amount of free FD10 and 10 equivalents of mBSA. Injected worms are incubated for 3 h.

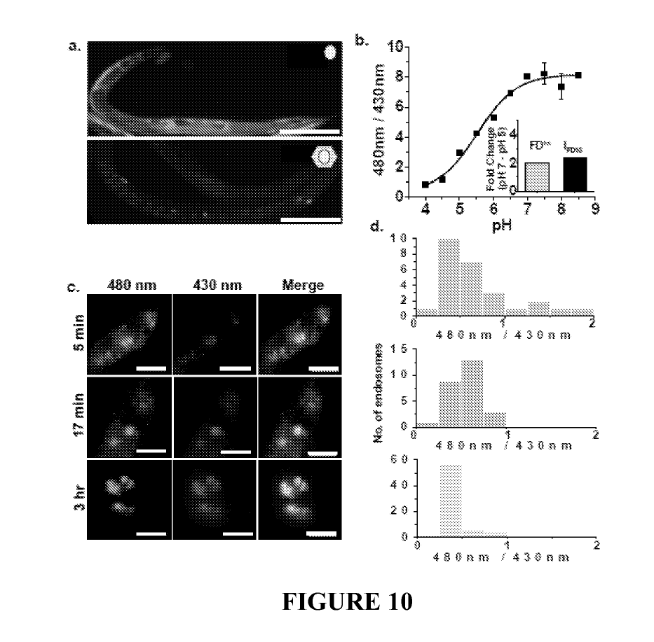

When 15 μM free FD10 is microinjected into C. elegans, it is clear that there is highly non-specific distribution of FD10 throughout the pseudocoelom (FIG. 10a, upper panel). Injection of the same concentration of FD10 encapsulated in the host-cargo complex I_{FD10} (DNA: 3 μm, FD10: 15 μM), in complete contrast, shows specific uptake only by the coelomocytes (FIG. 10a, lower panel and FIG. 11). This is completely abolished when I_{FD10} is co-injected with mBSA (FIGS. 11 and 12).

### Example 9

### Functionality of the Cargo of I_{FD10} In Vivo

In order to confirm that the cargo (FD10) is still functional post-encapsulation within the DNA host and post-delivery in vivo, the pH of I_{FD10} containing compartments inside the coelomocytes is measured. Fluorescein is a well known fluorescent reporter of pH and intracellular pH measurements use dual excitation of fluorescein at 480 nm and 430 nm.

### In Vivo pH Clamping

For the in vitro calibration curve, 150 nM of I_{FD10}, dissolved in 20 mM pH clamping buffer of a given pH (120 mM KCl, 5 mM NaCl, 1 mM CaCl₂, 1 mM MgCl₂, 20 mM HEPES), is excited at 480 nm (±10 nm) and 430 nm (±15 nm) and emission measured at 535 nm (±15 nm). The fluorescent ratios obtained are plotted against pH values to obtain a calibration curve. For the in vivo pH calibration in coelomocytes, hermaphrodites are injected with 3 μM of I_{FD10} and incubated for 3 h. After piercing the cuticle with a microinjection needle, the worms are immersed in pH clamping buffer of desired pH containing 100 μM of nigericin for 75 mins. Coelomocytes are imaged after mounting the worms on a 2% agarose pad and anesthetizing using 40 mM sodium azide in M9 buffer.

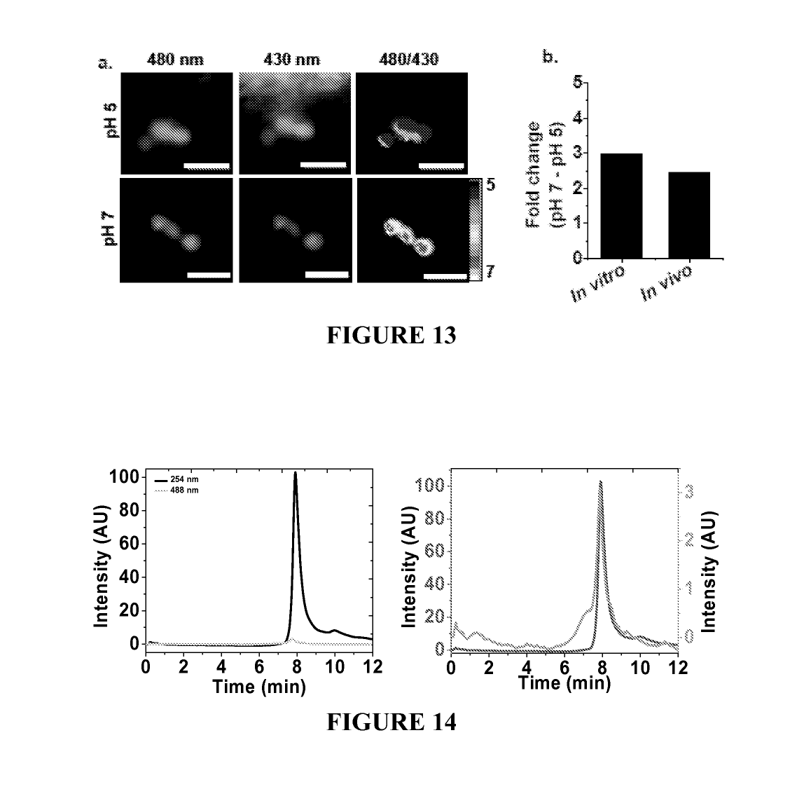

As mentioned above, the in vitro pH calibration curve for I_{FD10} is generated for pH ranging from about 4 to 8 (FIG. 10b). Further, the I_{FD10} complex is microinjected in worms and these worms are immersed in pH clamping buffers containing the ionophore nigericin at pH between 7 and 5 for 75 minutes. This clamps the pH of the endosomes in coelomocytes to the pH of the external buffer (FIG. 13a). The coelomocytes are imaged using dual excitation method. The fold-change in 480/430 emission ratio of both free and encapsulated FD10 remains unaltered in vivo (Inset, FIG. 10b). Also, the fold change in 480/430 emission ratio for I_{FD10} remains unchanged in vitro as well as in vivo (FIG. 13b). Cumulatively, these indicate that the functionality of FD10 post-encapsulation is quantitatively preserved. The functionality of the encapsulated FD10 in living worms under native conditions is demonstrated by the ability of I_{FD10} to map pH changes during endosomal maturation along the ALBR pathway in coelomocytes. The 480/430 ratio of ˜35 endosomes in coelomocytes that are marked by I_{FD10} at 5 min, 17 min and 3 h post injection (FIG. 10c) gave a distribution as shown in FIG. 10d. The in vivo pH of compartments containing I_{FD10} at 3 h post injection gave a 480/430 ratio of 2.5 that corresponded to a pH of ˜5.2. This is consistent with I_{FD10} residing predominantly in the lysosomes, at 3 h, that are shown to have pH ˜5.0.

Further, the FD10 measures the pH inside the coelomocytes even while it is encapsulated in the DNA host. The DNA icosahedron has pores of about 3 nm. Hence, small molecules, ions and protons in the coelomocytes or other biological systems easily diffuse in and out of the icosahedron enabling the FD10 molecules to sense the protons and measure the pH of coelomocytes even in the encapsulated state. This aspect adds to the advantage of the instant disclosure, as it does not require an additional step for the release of the cargo FD10 from the DNA host. Thus the cargo that is pH sensitive can measure the biological concentrations of these molecules and can give out a signal without the need to release it.

On the other hand, for the functioning of a completely closed host cargo system which has neutral bioimaging cargo encapsulated within it, will need to be delivered to a particular site, then release the encapsulated cargo so that the cargo initiates the bioimaging.

Further, the instant method offers another advantage. Since the cargo is not released from the DNA icosahedron, it performs bioimaging for longer periods of time till the host is stable. The host is found to be stable for a minimum period of about 24 hrs, thus bioimaging can be done throughout the day.

Image Acquisition and Quantification

In the above experiment, in cellulo and in vivo imaging employed TE2000U microscope (Nikon, Japan), equipped with mercury arc illuminator (Nikon, Japan), Photometrics Cascade II CCD camera (Tucson, Ariz.), and Chroma dichroic and band pass filters (USA). Images in each experiment are acquired under the same settings, using Metamorph software (Universal Imaging, PA). In vivo colocalization imaging is performed on Fluoview 1000 confocal microscope (Olympus, Japan) equipped with argon ion laser for 488 nm excitation and He—Ne laser for 543 excitation with dichroics, excitation, and emission filters suitable for each fluorophore. Image quantification is done using ImageJ ver. 1.42 software (NIH). Briefly, hemocyte images are background subtracted, cell boundary is determined from the bright field image and total cell intensity calculated. The mean intensity in five fields of view is obtained and normalized with respect to I_{FD10} alone. For the in vivo uptake assays, the number of coelomocytes labeled are counted and expressed as a percentage of the total number of coelomocytes. For in vivo pH measurements, dual excitation of I_{FD10} is performed. After background subtraction, endosomes are demarcated in each image and their mean intensity calculated. The ratio of mean intensities at 520 nm when excited at 480 nm as well as 430 nm gives the 480/430 ratio of each endosome.

### Example 10

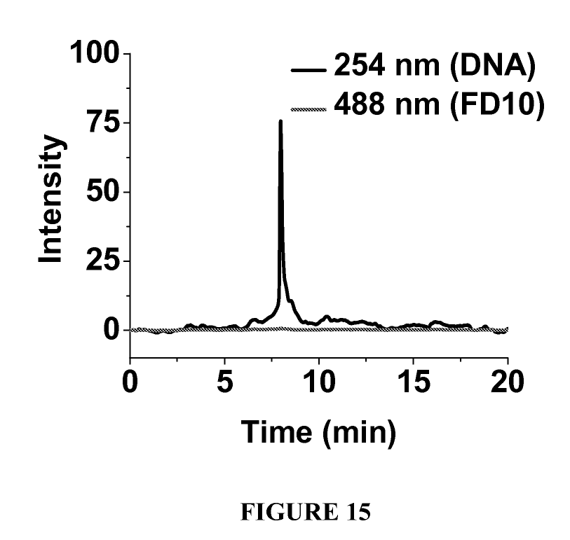

In order to determine the effect of concentration on encapsulation of neutral bio-imaging agents, encapsulation of FD10 within DNA icosahedra is done, wherein the concentration of DNA icosahedra and FD10 are 100 nM and 400 nM respectively or in the ratio of 1:4. DNA icosahedron and FD10 are mixed/annealed at concentration ratios of about 100 nM:400 nM to form I_{FD}10 Complex. The complex formed is purified from 0.8% agarose gel run in 1×TAE buffer and loaded on HPLC. Upon analysis of the chromatogram obtained (FIG. 15), it is found that the peak corresponding to only empty DNA icosahedron (absorbance at 254 nm) is detected on the usage of the above concentration ranges whereas, no peak corresponding to presence of FD10 (absorbance at 488 nm) is detected. This clearly indicates that at low concentration ratio, the DNA icosahedron is unable to encapsulate FD10.

Encapsulation of neutral bioimaging molecules within the DNA cage without any molecular interaction between them is achieved when the cargo is used in excess. The concentration regime that is used for encapsulation of neutral bioimaging agents such as FD10 and TD10 is about 1 mM to about 4 mM. Thus for FD10 encapsulation, the concentrations used are DNA: 1 micromolar and FD10: 2 millimolar which means cargo is at least 2000 fold excess present in solution. It is necessary to use concentration of molecule such that there is at least one molecule of cargo per 1000 nm³ volume which is the encapsulable volume of the icosahedron.

The HPLC trace of the icosahedron has been given (FIG. 14) which is in the concentration of 1 μM carrying FD10 within it. From the intensities of peaks at 254 and 488 nm, it is found that an average of 2 molecules of FD10 are encapsulated within it. However, even at this concentration, during the process of icosahedron formation, there may be 2 or 3 FD10 molecules which are encapsulated from solution to within the DNA icosahedron. This is because the final step of FD10 encapsulation within icosahedron takes about 6-8 hours annealing during which it is always possible that 2 or 3 FD10 molecules will be encapsulated. The numbers of cargo encapsulated is increased, if about 4 to about 5 mM FD10 is used, which leads to fully occupying the volume of icosahedron. However, increasing beyond this concentration of neutral bioimaging molecules, does not add to number of encapsulated molecules within DNA icosahedron.

### Example 11

Encapsulation of neutral molecules within DNA icosahedron without interactions will require cargo to be used in high concentrations. Similar behaviour is observed for another neutral cargo—TD10 (tetrmethyl rhodamine dextran, 10 kDa). TMR dextran behaves exactly similar to FITC dextran, since the cargo is same—dextran. Only the fluorophores are changed.

As illustrated in FIG. 6, the anisotropic behaviour or I_{FD10} is found to be the same as that for I_{FD10}. Thus, FD10 and TD10 show the same trend of association with DNA icosahedron. Further, FD10 in the IFD10 complex is not associated with the DNA scaffold, I Fluorescence anisotropies of two different fluorophores when encapsulated inside or covalently attached to the DNA icosahedron and its component modules show similar trends, indicating that anisotropy observations are fluorophore independent.

Conclusion:

The present disclosure demonstrates the encapsulation of a functional neutral bioimaging molecule as a cargo within a synthetic DNA host capsule of well-defined structure. Encapsulation to form a synthetic host-cargo complex is a molecular recognition-free strategy as is described in the present disclosure. Encapsulation relies mainly on size compatibility between the host and cargo (Table 2). The encapsulation studies indicate that for a cargo such as FD10, of size 5.2 nm (i) the presence of excess FD10 does not impede the formation of DNA icosahedra in solution and (ii) the FD10 molecules are associated with the capsules in a manner that does not significantly alter the size of the icosahedral DNA capsule. Post-encapsulation, quenching studies reveal that the FD10 present within the DNA cage is inaccessible to particles greater than 3 nm in size, completely accessible to particles below 2 nm, while particles between 2-3 nm have limited access. Such size dependent quenching has been observed in the case of the fluorophores present inside the endoplasmic reticulum (ER) where the transition point corresponds to the average pore size of the translocon, the protein conduction channel present on the ER. Given that the pore size of I has been shown to be ˜2.5 nm experimentally and 2.8 nm by a theoretical model, these studies confirm that FD10 is encapsulated as cargo within the DNA icosahedron as a host in the I_{FD10} complex.

Further, concentrations of the molecules which are encapsulated in DNA icosahedra are also dependent on the size of the molecule. Hence, as illustrated in the table 2, the size of cargo is also a limiting factor and it is found that cargoes smaller than 10 nm are be encapsulated within DNA icosahedron while the ones larger than 10 nm cannot be encapsulated.

Anisotropy studies to probe the mobility of the encapsulated cargo within the DNA host showed only a very minor increase in anisotropy between free and encapsulated FD10. Such a meager increase could also arise from weak interactions between FD10 and I that could be captured by measuring the probe lifetime of the cargo due to the quenching ability of the DNA host. However, free FD10 and I_{FD10} showed similar fluorescence lifetimes within experimental error (Inset, FIG. 3b), confirming that anisotropy clearly reports on the motion of the probe. This reveals that any potential weak interactions between FD10 and DNA scaffolds are not responsible for the mild increase in anisotropy seen in the host-cargo complex. These are complemented by studies on the rotational correlation times between free and encapsulated cargo. Here too, only a very small change in rotational correlation time (θ) of ˜1 ns between free and encapsulated FD10 is observed, revealing that the entrapped FD10 molecule is very weakly associated with the synthetic icosahedral DNA host, I. Cumulatively, the studies on anisotropy and rotational correlation times suggest a model where the FD10 encapsulated inside the central void experiences only slightly hindered rotation due to its confined environment (FIGS. 3c, 3d). In Drosophila hemocytes and many other cell types, free FD10 does not engage any membrane receptor and is therefore endocytosed only along with the external bulk solution, thus marking the fluid phase (FIG. 7a). mBSA is a polyanionic molecule that is endocytosed exclusively by the ALBR pathway and thus does not compete out the uptake of free FD10 due to their uptake by completely independent pathways. The studies of the instant disclosure have established that DNA structures are endocytosed via the Anionic Ligand Binding Receptor (ALBR) pathway in Drosophila hemocytes, and are efficiently competed out by mBSA. Studies on the endocytic uptake of encapsulated FD10 in Drosophila hemocytes reveal that it is uptaken exclusively by the ALBR pathway, showing a dramatic change in uptake properties from free FD10 that is internalized by the fluid phase pathway. It is notable that without any chemical modification per se to the cargo, and mere physical encapsulation within a synthetic DNA host, an entirely new set of biochemical interactions is made available to the cargo resulting in a complete alteration of its natural endocytic property. The amplification of such a small difference in chemical terms to the cargo into an incommensurable difference in the host-cargo complex in terms of biochemical interactions in the context of a more complex system is the hallmark of emergent behavior.

This altered endocytic behavior shown by the DNA host-cargo complex is also borne out in vivo. Coelomocytes of C. elegans express anionic ligand binding receptors that internalize negatively charged molecules such as DNA by receptor mediated endocytosis. Competition experiments of I_{FD10} uptake in coelomocytes of C. elegans involving co-injection of I_{FD10} with excess of mBSA clearly revealed that it is uptaken via the ALBR pathway as against fluid phase endocytosis for free FD10 (FIG. 12). Here again the DNA host-cargo complex shows a key signature of emergent behavior, that in the context of a more complex system, new properties emerge that could not be predicted in a simpler system. Thus, not only is the altered endocytic pathway manifested in vivo, but delivering the cargo to only those cells of the organism that possess the relevant receptor is also observed. This delivery behavior and hence uptake of I_{FD10} is abolished when co-injected with mBSA that competitively binds to the relevant receptors (FIG. 12), demonstrating control over the cargo by manipulation of the synthetic host-organism interactions.

Cargo functionality post-encapsulation and post-delivery into endosomes on the ALBR pathway in coelomocytes of C. elegans is checked by assessing its ability to map spatiotemporal pH changes associated with maturation of the endosomes that it marks. pH clamping of coelomocytes labeled with I_{FD10} at pH 5 and 7 showed that the in vivo fold change in 480/430 emission of the cargo is unchanged from the in vitro values. A time-dependent study on endosomal pH values showed characteristic, progressively narrowing pH distributions accompanied by acidification as the endosomes matured from the early endosomes to late endosomes to the lysosome, where the pH is tightly regulated. FD10 has two properties, one of non-targeted endocytic uptake by the fluid phase and another of pH sensitivity. It is important to note that emergent behavior is observed in the case of the former but not in the latter. The present disclosure presents the in vivo observation of predicted emergent behavior that has been the driving force for the construction of synthetic host-cargo systems.

TD10 can also be used for bioimaging along similar lines as used for FD10. However, since TD10 is a pH insensitive neutral bioimaging molecule, it can be used for visualizing biological phenomenon such as endocytosis systems.

Applications:
a. Various neutral bioimaging molecules can be encapsulated like high performance imaging devices like neutral fluorescent probes, neutral fluorescent proteins, etc. Functional Bio-imaging. The structures disclosed in the present disclosure are used to encapsulate various functional fluorescent probes for bio-imaging in vivo. The probes like Fluorescent Dextrans (FITC and TMR dextrans), peptides, inorganic nanoparticles, fluorescent nanoparticles, magnetic nanoparticles, fluorescent proteins, PET imaging probes, radioactive probes, Raman active probes, functional proteins like enzymes are encapsulated inside these polyhedra and these systems are used for functional bio-imaging using various techniques like microscopy, Raman imaging, MRI, electron microscopy, etc. The above mentioned probes fall in the size regime of 3-10 nm which is most appropriate for encapsulation inside DNA icosahedron. Also, the size of DNA icosahedron is enlarged depending on the probe size to be encapsulated.
b. These structures can be used as containers to study the functional behavior of encapsulated molecules in confined environments.
c. The present disclosure can be employed as bioimaging agents and as DNA containers for precise control over the reactivity of encapsulated molecules.

We claim:

1. A method of delivering neutral bio-imaging molecule(s) to a cell, said method comprising act of encapsulating the neutral bioimaging molecule(s) within a DNA icosahedron and delivering the DNA icosahedron to the cell.

2. The method as claimed in claim 1, wherein the encapsulating of the neutral bio-imaging molecule(s) within the DNA icosahedron comprises acts of:
c) assembling DNA molecules to obtain a semi-icosahedral DNA capsule; and
d) incubating the neutral bio-imaging molecule(s) with the semi-icosahedral DNA capsule, and ligating the semi-icosahedral DNA capsules to obtain neutral bio-imaging molecule(s)-DNA icosahedron complex, wherein the DNA icosahedron encapsulates the neutral bio-imaging molecule(s).

3. The method as claimed in claim 1, wherein the neutral bio-imaging molecule is selected from a group comprising Fluorescent Dextrans preferably FITC Dextran and TMR Dextran, peptides, inorganic nanoparticles, fluorescent nanoparticles, magnetic nanoparticles, fluorescent proteins, PET imaging probes, radioactive probes, Raman active probes and functional proteins or any combination thereof.

4. The method as claimed in claim 1, wherein the neutral bio-imaging molecule is at concentration ranging from about 0.5 mM to about 5 mM.

5. The method as claimed in claim 2, wherein the assembling is carried out by associating DNA junction selected from a group comprising V junction, U junction and L junction or a combination thereof to form semi-icosahedral DNA capsule.

6. The method as claimed in claim 2, wherein the incubating is carried out at pH ranging from about 6 to about 8, preferably about 7, at a temperature ranging from about 4° C. to about 55° C., preferably about 45° C., and for a time duration ranging from about 3 hours to about 5 hours, preferably about 4 hours.

7. The method as claimed in claim 2, wherein the DNA icosahedron encapsulating the neutral bioimaging molecule(s) is at concentration ranging from about 0.5 mM to about 5 mM and wherein the DNA icosahedron has pore size ranging from about 2 nm to about 3 nm, preferably about 2.8 nm.

8. The method as claimed in claim 2, wherein the ligating is carried out using chemicals selected from a group comprising N-Cyano Imidazole [NCI] and Cyanogen Bromide, preferably N-Cyano Imidazole [NCI]; at temperature ranging from about 15° C. to about 25° C., preferably about 20° C.

9. The method as claimed in claim 2, wherein re-incubation is carried out after the ligation for time duration ranging from about 24 hours to about 96 hours, preferably about 72 hours and at temperature ranging from about 0° C. to about 20° C., preferably about 4° C.

10. The method as claimed in claim 1, wherein the delivery is selected from a group comprising in-vivo, ex-vivo and in-vitro delivery.

11. The method as claimed in claim 1, wherein the in-vivo delivery is carried out by microinjecting the DNA icosahedron encapsulating the neutral bioimaging molecule(s); and wherein ex-vivo and in-vitro delivery is carried out by electroporation or pulsing the cell with the DNA icosahedron encapsulating the neutral bioimaging molecule(s).

12. The method as claimed in claim 1, wherein after the delivery, the DNA icosahedron encapsulating the neutral bioimaging molecule(s) is taken up by the cell through interaction of the cell receptors with the DNA icosahedron by pathways selected from a group comprising receptor mediated endocytosis, anionic ligand-binding receptor (ALBR) pathway, recycling pathways and fluid phase uptake pathway, preferably ALBR pathway.

13. A complex comprising DNA icosahedron encapsulating neutral bioimaging molecule(s).

14. The complex as claimed in claim 13, wherein the complex delivers the neutral bio-imaging molecule(s) to a cell.

15. The complex as claimed in claim 14, wherein the delivery is selected from a group comprising in-vivo, ex-vivo and in-vitro delivery.

16. The complex as claimed in claim 15, wherein the in-vivo delivery is carried out by microinjecting the DNA icosahedron encapsulating the neutral bioimaging molecule(s); and wherein ex-vivo and in-vitro delivery is carried out by pulsing the cell with the DNA icosahedron encapsulating the neutral bioimaging molecule(s).

17. The complex as claimed in claim 13, wherein the neutral bio-imaging molecule is selected from a group comprising Fluorescent Dextrans preferably FITC Dextran and TMR Dextran, peptides, inorganic nanoparticles, fluorescent nanoparticles, magnetic nanoparticles, fluorescent proteins, PET imaging probes, radioactive probes, Raman active probes and functional proteins or any combination thereof.

18. The complex as claimed in claim 13, wherein the neutral bio-imaging molecule is at concentration ranging from about 0.5 mM to about 5 mM.

19. The complex as claimed in claim 13, wherein the neutral bio-imaging molecule is a polymer based fluorescent molecule, functioning as a pH reporter devoid of any molecular recognition within the DNA icosahedrons.

20. The complex as claimed in claim 13, wherein the DNA icosahedron encapsulating the neutral bioimaging molecule(s) is at concentration ranging from about 0.1 μM to about 3 μM and wherein the DNA icosahedron has pore size ranging from about 2 nm to about 3 nm, preferably about 2.8 nm.

21. A process for synthesising a complex comprising DNA icosahedron encapsulating neutral bioimaging molecule(s), said process comprising acts of:
c) assembling DNA molecules to obtain a semi-icosahedral DNA capsule; and
d) incubating the neutral bioimaging molecule(s) with the semi-icosahedral DNA capsule, and ligating the semi-icosahedral DNA capsules to obtain neutral bioimaging molecule(s)-DNA icosahedron complex, wherein the DNA icosahedron encapsulates the neutral bio-imaging molecule(s).
